(19) United States
(12) Patent Application Publication
(10) Pub. No.: US 2025/0032593 A1
(43) Pub. Date: Jan. 30, 2025

(54) EUKARYOTIC ELONGATION FACTOR-2 KINASE AS A CYTOTOXIC T LYMPHOCYTES BOOSTER

(71) Applicant: The Texas A&M University System, College Station, TX (US)

(72) Inventor: Jianxun Jim Song, Bryan, TX (US)

(73) Assignee: The Texas A&M University System, College Station, TX (US)

(21) Appl. No.: 18/716,884

(22) PCT Filed: Dec. 20, 2022
(86) PCT No.: PCT/US2022/053532

§ 371 Date: Jun. 5, 2024

Related U.S. Application Data
(60) Provisional application No. 63/291,462, filed on Dec. 20, 2021.

(51) Int. Cl. A61K 38/45

(52) U.S. Cl. CPC A61K 38/45; C12Y 207/1102

(57) ABSTRACT

In an embodiment, the present disclosure pertains to a method of reducing cytocidal activity of cells. In some embodiments, the method includes altering eukaryotic elongation factor-2 kinase (eEF-2K) in a subject and inducing a reduction of cytocidal activity of cytotoxic T cells (CTLs) in the subject. In some embodiments, the altering of eEF-2K in the subject can include a decrease of eEF-2K in the subject.

### CROSS-REFERENCE TO RELATED APPLICATIONS

This patent application claims priority from, and incorporates by reference the entire disclosure of, U.S. Provisional Application 63/291,462 filed on Dec. 20, 2021.

### STATEMENT REGARDING FEDERALLY SPONSORED RESEARCH

This invention was made with government support under LC210150 awarded by the Department of Defense. The government has certain rights in the invention.

### TECHNICAL FIELD

The present disclosure relates generally to eukaryotic elongation factor-2 kinase (eEF2K) and more particularly, but not by way of limitation, to eEF2K as a cytotoxic T lymphocytes (CTL) booster.

### BACKGROUND

This section provides background information to facilitate a better understanding of the various aspects of the disclosure. It should be understood that the statements in this section of this document are to be read in this light, and not as admissions of prior art.

Mounting evidence has shown that the metabolic status of immune cells, as well as tumor cells, can greatly impact antitumor immunity. Immune activation, acquisition of effector functions, and generation of immune memory are all closely associated with alterations in cellular metabolism. A few connections between metabolic reprograming and T cell differentiation, survival, and function have been recently been reported. However, the precise molecular mechanisms and pathways involved remain to be fully elucidated.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In an embodiment, the present disclosure pertains to a method of reducing cytocidal activity of cells. In some embodiments, the method includes altering eukaryotic elongation factor-2 kinase (eEF-2K) in a subject and inducing a reduction of cytocidal activity of cytotoxic CD8⁺ T cells (CTLs) in the subject. In some embodiments, the altering of eEF-2K in the subject can include a decrease of eEF-2K in the subject.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIGS. 1A-1H illustrate how loss of eEF-2K alters the survival, proliferation, and function of CD8⁺ T cells. CD8⁺ T cells from wild type (WT) or eukaryotic elongation factor-2 kinase (eEF-2K) knockout (KO) C57BL/6 mice were activated with anti-CD3/CD28 antibodies. The percent survival, cytokine production, and proliferation were assessed. FIG. 1A shows the percent survival. FIG. 1B shows proliferation by carboxyfluoroscein succinimidyl ester (CFSE)-based flow cytometric-staining and mass spectrometric proteomics analysis of Ki-67. FIG. 1C shows enzyme-linked immunosorbent assay (ELISA) of IL-2 production on day 1 and day 3 post-stimulation. FIG. 1D shows graphical representation of carcinoembryonic Ag-related cell adhesion molecule 1 (CEACAM-1) expressing cells/field and independent replicates of flow cytometric analysis of IL-6 cytokine. FIG. 1E shows flow cytometric histogram analysis of CD27 co-stimulatory marker. FIG. 1F shows graphical analysis of independent replicates of CD27 expression derived from flow cytometric dot-plot analysis. FIG. 1G show flow cytometric histogram representation of CD28 co-stimulatory marker. FIG. 1H shows graphical analysis of independent replicates of CD28 expression derived from flow cytometric dot-plot analysis. Data shown are representative values derived from 3 identical and independent experiments. *p<0.05; **p<0.01; ***p<0.005; ****p<0.0001, unpaired t-test.

FIGS. 2A-2D illustrate CD8⁺ T cells deficient in eEF-2K are more metabolically active than WT CD8⁺ T cells. CD8⁺ T cells were isolated from WT or eEF-2K KO C57BL/6 mice. The CD8⁺ T cells were subsequently purified and activated with anti-CD3/CD28 antibodies and supplemented with IL-2 cytokine and grown for 3 to 4 days. The WT or eEF-2K KO CD8⁺ T cells were then analyzed for the extracellular acidification rate (ECAR) using Seahorse XFe96 analysis as per the manufacturer's protocol. FIG. 2A shows comparative ECAR of eEF-2K KO CD8⁺ T cells compared to that of WT CD8⁺ T cells on day 2 post activation with anti-CD3/CD28 antibodies. FIG. 2B shows liquid chromatography with tandem mass spectrometry (LC/MS-MS) proteomics spectral analysis shown as bar-graphs of intermediates of glycolysis pathway from day 3 post-activated CD8⁺ T cells. FIG. 2C shows day 4 post-activation ECAR analysis of WT vs eEF-2K KO CD8⁺ T cells. FIG. 2D shows a schematic representation of the glycolytic pathway showing the enzymes upregulated in CD8⁺ T cells deficient in eEF-2K in red color. Data shown are representative values derived from 3 independent experiments. *p<0.05; **p<0.01; ***p<0.005, unpaired t-test.

FIGS. 3A-3F illustrate loss of eEF-2K upregulates the Akt-mTOR signaling and HSP90 in CD8⁺ T cells. FIG. 3A shows quantification of western blot analysis of upregulation of Akt-pAkt proteins using Image J software of independent experiments. FIG. 3B shows p-RPS6kb was validated by flow cytometric analysis. FIG. 3C shows that loss of eEF-2K upregulates the mTOR signaling pathway. Independent blots were quantified with Image J software and normalized to β-actin. FIG. 3D shows a seahorse metabolic profile shows that the dysfunctional metabolism in eEF-2K KO CD8⁺ T cells is partially restored to WT levels on treatment with rapamycin. FIG. 3E shows survival of eEF-2K KO CD8⁺ T cells was partially restored to WT levels on treatment with rapamycin. FIG. 3F shows the LC-MS/MS spectra indicated that all major heat shock proteins were upregulated in eEF-2K KO CD8⁺ T cells compared to their WT counterparts with significant upregulation of HSP90-aa1 and HSP90-ab1. mTORi=Rapamycin. Data shown are representative values derived from 3 independent experiments. *p<0.05; **p<0.01, unpaired t-test.

FIGS. 4A-4C illustrate loss of eEF-2K in CAR-CD8⁺ T cells impairs their ability to infiltrate tumor tissues. On Day 28 post-induction of tumors, the explanted tumor was analyzed. FIG. 4A shows graphical representation of tumor-infiltrating lymphocytes (TILs) derived from flow cytometric analysis. FIG. 4B shows IL-6 and CEACAM-1 expression of TILs by flow cytometric analysis. FIG. 4C shows quantification of different immune cell populations.

FIGS. 5A-5B illustrate loss of eEF-2K results in CD8⁺ T cell exhaustion. WT and eEF-2K KO CD8⁺ T cells were activated using anti-CD3/anti-CD28 Abs for six days. FIG. 5A shows flow cytometric analysis was performed to assess expression of PD-1 and CD62L. The graphical representation of dot-plot analysis in triplicates is shown. FIG. 5B shows graphical representation of flow cytometric assay for CD27 and CD28 on days 1 and 3.

FIG. 6 illustrates upregulation of PD-1 and Tim-3 in eEF-2K KO CD8⁺ T cells can be abrogated by rapamycin (mTOR-i). Flow cytometric analysis of PD-1 and Tim-3 expression in WT CD8⁺ T cells, eEF-2K KO CD8⁺ T cells and eEF-2K KO CD8⁺ T cells treated with rapamycin (mTOR-i). Data shown are the graphical representation of the data of 3 independent experiments.

FIGS. 7A-7B illustrate loss of eEF-2K compromises the cytotoxicity of eEF-2KO CD8⁺ T cells. Control MC38 cells or the MC32CEA Ag-expressing cells were co-cultured with WT or eEF-2K 681 KO CD8⁺ T cells, and then their cytotoxicity was assessed. FIG. 7A shows graphical representation of representative dot-plots obtained from flow-cytometric analysis. FIG. 7B shows graphical representation of lactate dehydrogenase (LDH) release assay of MC32CEA colon cancer cells co-cultured with WT or eEF-2K KO CD8⁺ T cells.

FIGS. 8A-8I illustrate expression of costimulatory and exhaustion markers by the CEA-specific CAR-CD8⁺ T cells with or without eEF-2K. On day 28 after adoptive transfer, the WT or eEF-2K KO Thy 1.2 CD8⁺ T cells (FIG. 4A-FIG. 4C) were analyzed for the expression of costimulatory and exhaustion markers, using flow cytometry. FIG. 8A shows graphical representation of CD27 expression derived from flow cytometric dot plots. FIG. 8B shows graphical representation of CD28 expression derived from flow cytometric dot plots. FIG. 8C shows graphical representation of TIM3 expression derived from flow cytometric dot plots. FIG. 8D shows graphical representation of PD1 expression derived from flow cytometric dot plots. The intracellular cytokine expression was also assessed from the WT or eEF-2K KO Thy1.2 CD8⁺ T cells after re-stimulating the cells ex-vivo with anti-CD3/CD28 antibodies. FIG. 8E shows graphical representation of IL-2 cytokine expression derived from flow cytometric dot plots. FIG. 8F shows graphical representation of IL-1α cytokine expression derived from flow cytometric dot plots. FIG. 8G shows graphical representation of IL-4 cytokine expression derived from flow cytometric dot plots. FIG. 8H shows graphical representation of IFN-γ cytokine expression derived from flow cytometric dot plots. FIG. 8I shows graphical representation of TNF-α cytokine expression derived from flow cytometric dot plots. Graphical data shown are mean±standard deviation (SD) from values derived from 3 different wells of 3 independent experiments and statistically analyzed using student's t-test (*p<0.05; **p<0.01; ***p<0.005; ****p<0.0001).

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

Eukaryotic elongation factor-2 kinase (eEF-2K) has important roles in stress responses and cellular metabolism. Cytotoxic T cells such as CD8⁺ T cells from eEF-2K KO (knockout) mice were more proliferative but had lower survival than their wild-type counterparts after their activation, followed by occurrence of premature senescence and exhaustion. eEF-2K KO CD8⁺ T cells were more metabolically active and showed hyperactivation of the Akt-mTOR-S6K pathway. Loss of eEF-2K significantly impaired the activity of CD8⁺ T cells. Furthermore, the antitumor efficacy and tumor infiltration of the CAR-CD8⁺ T cells lacking eEF-2K were remarkably reduced as compared to the control CAR-CD8⁺ T cells. Thus, eEF-2K is critically required for sustaining the viability and function of cytotoxic CD8⁺ T cells, and therapeutic augmentation of this kinase may be exploited as a novel approach to reinforcing CAR-T therapy against cancer.

eEF2K mediates T cell function by controlling mitochondrial activities and regulating the activity of the transcription factor STAT3. More significantly, absence of eEF2K can aggravate autoimmune colitis and arthritis development through the increased production of inflammatory cytokines and the development of Th17 cells. Therefore, targeting eEF2K may provide new insights into the improvement of the efficacy of T cell therapy in inflammation and autoimmunity.

Successful chimeric antigen (Ag) receptor (CAR) T-cell therapy for cancer encounters several barriers, including insufficient amounts of tumor Ag-specific T cells due to clonal erasure, poor activation of T cells, accumulation of tolerogenic Ag-presenting cells in the tumor microenvironment (TME), and formation of an immunosuppressive TME. Mounting evidence has shown that the metabolic status of immune cells as well as tumor cells can greatly impact antitumor immunity. Immune activation, acquisition of effector functions and generation of immune memory are all closely associated with alterations in cellular metabolism. A few connections between metabolic reprograming and T cell differentiation, survival, and function have been reported recently. However, the precise molecular mechanisms and pathways involved remain to be fully elucidated.

eEF-2K, a member of the atypical α-kinase family, is an evolutionarily conserved regulator of protein synthesis. This kinase phosphorylates eEF-2, a 100 kDa protein that promotes ribosomal translocation from the A to the P-site and induces movement of mRNA along the ribosome during translation. Phosphorylation of eEF-2 on Thr56 by eEF-2K terminates peptide elongation by decreasing the affinity of this elongation factor for the ribosome. Several studies have demonstrated that various stress factors such as growth factor deprivation, nutrient deficiency, and oxidative and chemical insults are potent stimulators of eEF-2K. Moreover, the activity of this kinase is critically required for survival of stressed cells. It has also been reported that eEF-2K plays a crucial role in regulating autophagy and cellular ATP in tumor cells, and in promoting the Warburg effect. eEF-2K has a critical role in determining the fate, function, and antitumor immunity of cytotoxic T cells. Using T cells from eEF-2K knockout (KO) mice, it is demonstrated that loss of eEF-2K significantly reduces the survival and function of cytotoxic CD4⁺ and CD8⁺ T cells (CTLs), and this is associated with altered cell proliferation, premature cellular senescence, and exhaustion, activated Akt-mTOR-S6K signaling and reprogrammed metabolism. These findings may have important implications in developing more effective strategies to improve CAR-T therapy for cancer.

CD4⁺ T cells deficient in eEF2K differentiate into Th17 cells with dysfunctional phenotypes. The number of IL-17A-producing cells markedly increases in eEF2K KO cell cultures after cytokines activation with IL-4, IL-6, IL-23, TGF-β and IFN-γ. The expression of IL-23R, one of the signatures of Th17 cell surface markers, shows an increase in eEF2K KO Th17 cells. This indicates an essential role of eEF2K in maintaining cytotoxic T cell activities, including survival, proliferative capacity, and senescence.

CD4⁺ T cells undergo metabolic reprogramming and multiple biological processes to satisfy their energetic and biosynthetic demands throughout their lifespan. Several of these metabolic pathways result in the generation of reactive oxygen species (ROS). The imbalance between ROS generation and scavenging results in severe damage to the cells and potential cell death, ultimately leading to T cell-related diseases. Reactive Oxygen Species (ROS) produced within the mitochondria are required for CD4⁺ T cell functions and Th17 differentiation. An increased intracellular ROS production was detected in eEF2K KO CD4⁺ T cells, compared to WT CD4⁺ T cells, which indicates that eEF2K regulates T cell function through ROS production.

STAT3 promotes IL17A cytokine secretion in eEF2K deficient CD4⁺ Th17 cells. STAT3, one of the primary transcription factors of Th17s and a regulator of the mitochondrial respiratory chain, is expressed at higher levels in eEF2K KO CD4⁺ T cells compared to WT CD4⁺ T cells. eEF2K coordinately regulates CD4⁺ T cells function by modulating a feedback loop between ROS to STAT3. eEF2K KO CD4 T cells upregulate STAT3 expression, resulting in mitochondrial dysfunction and redox imbalance, which further leads to pro-inflammatory Th17 differentiation and function. eEF2K mediates CD4 T cell function and differentiation in Th17 cells through the induction of IL-17A and IL-17F cytokines, via the STAT3-ROS signaling pathway.

Loss of eEF2K results in higher inflammation and increased severity of arthritis and colitis development. This is a result of Th17 cell differentiation resulting from the absence of eEF2K, which enhances STAT3 activation. eEF2K is functionally essential in inflammation and autoimmunity, including autoimmune arthritis and colitis, which serves as a new paradigm for treating inflammation and autoimmune diseases

### WORKING EXAMPLES

Reference will now be made to more specific embodiments of the present disclosure and data that provides support for such embodiments. However, it should be noted that the disclosure below is for illustrative purposes only and is not intended to limit the scope of the claimed subject matter in any way.

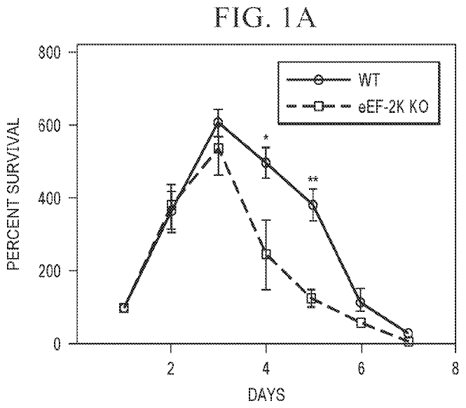

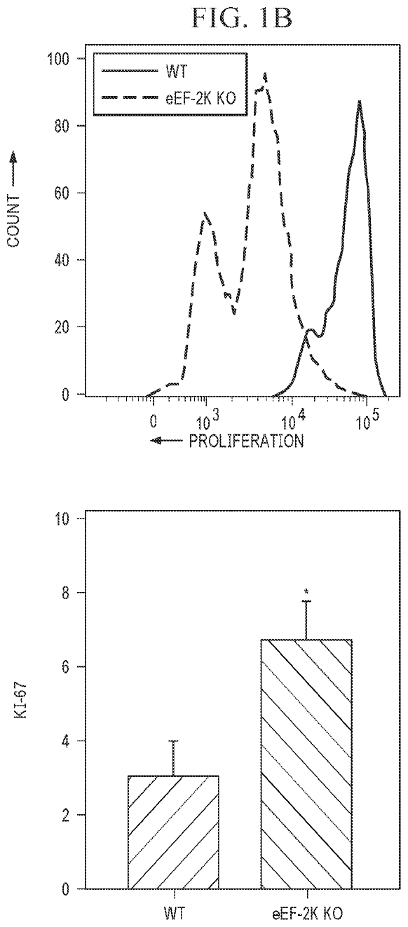

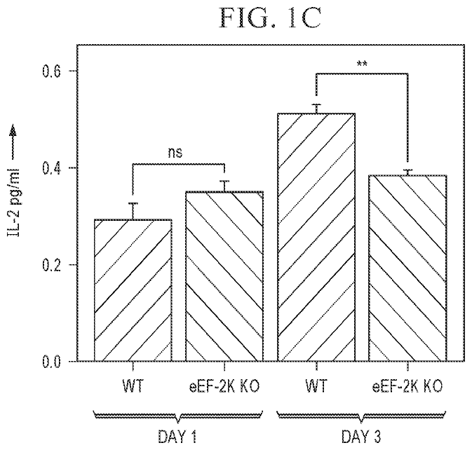

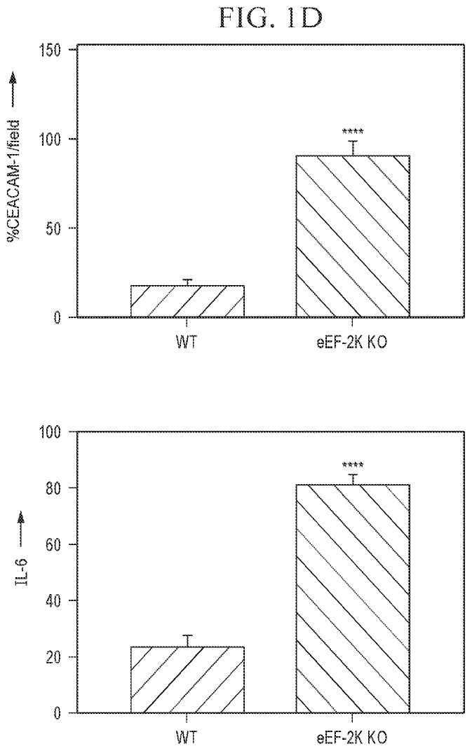

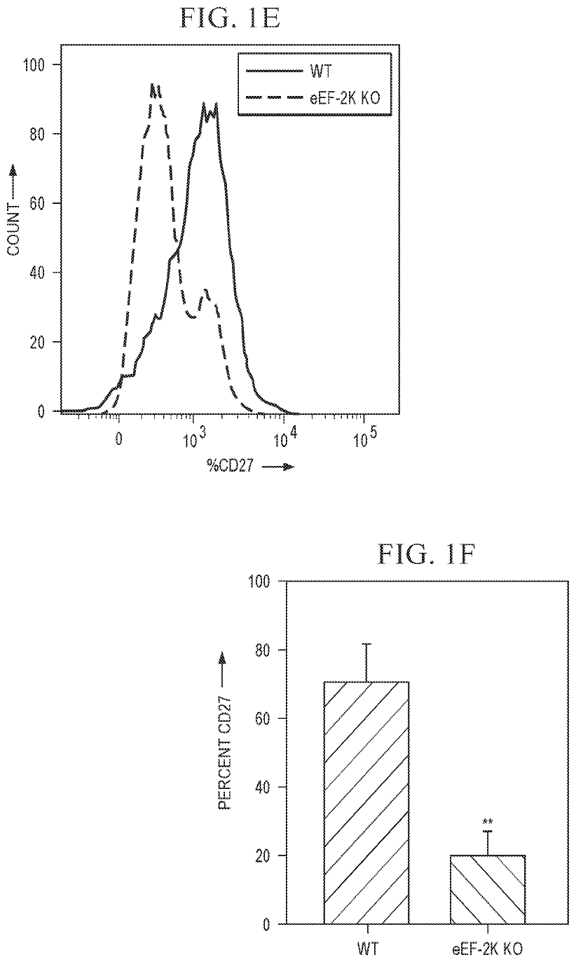

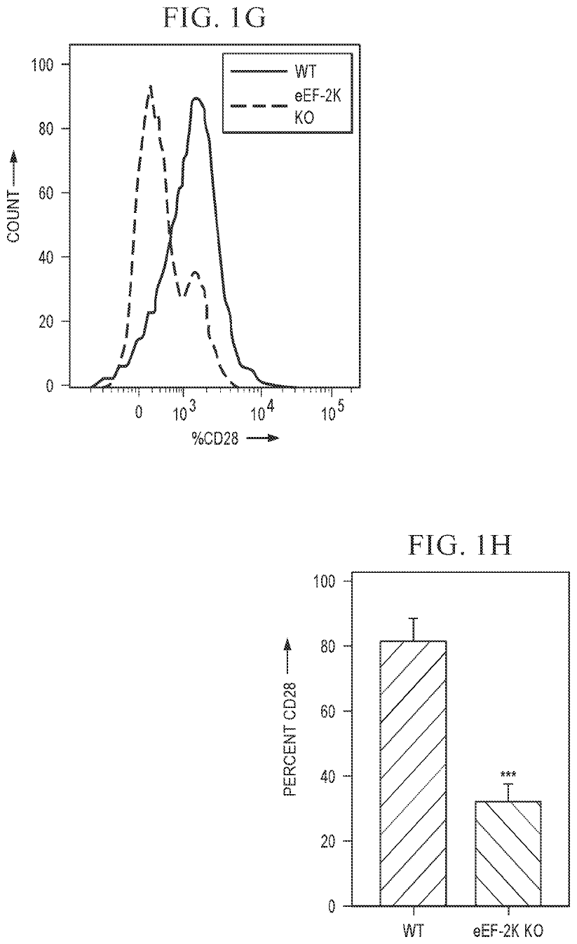

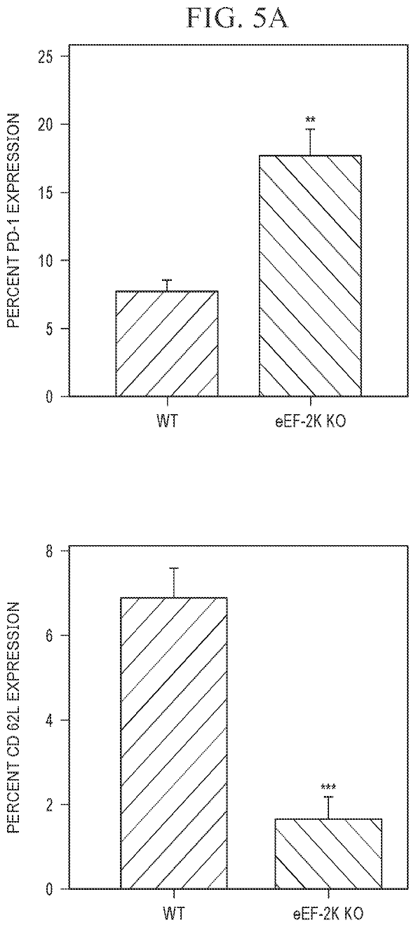

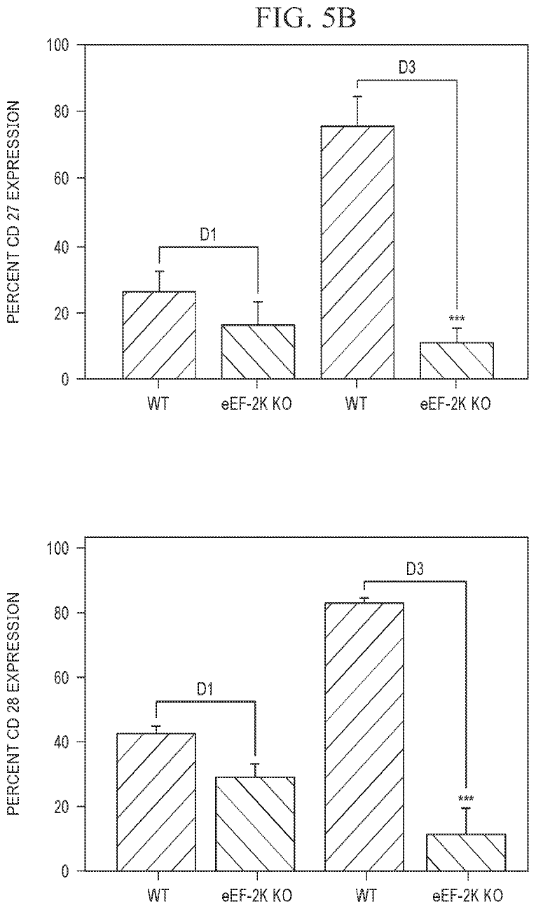

Loss of eEF-2K alters the fate and function of CD8⁺ T cells. To determine the effects of eEF-2K on CD8⁺ T cells, the T cells from either the wild-type (WT) or eEF-2K KO C57BL/6 mice were isolated, and then stimulated them with anti-CD3/CD28 antibodies. It was found that 3 days after stimulation, the survival of eEF-2K KO CD8⁺ T cells peaked slightly higher than that of WT CD8⁺ T cells, but was significantly lower than the controls (2-fold change; p<0.05) (FIG. 1A) on day 4, day 5, and day 6 post-stimulation; nevertheless, eEF-2K KO CD8⁺ T cells were more proliferative than WT CD8⁺ T cells after activation, as analyzed by CFSE-based flow cytometry and by Ki-67 expression (FIG. 1B). Correlatively, significantly decreased production of IL-2 was observed in eEF-2K KO CD8⁺ T cells compared to WT CD8⁺ T cells 3 days after their activation (FIG. 1C). In addition, 3-4 days after stimulation by anti-CD3/CD28 antibodies, a significantly greater amount of senescence associated β-galactosidase (SABG)-positive cells were found in the population of eEF-2K KO CD8⁺ T cells than in WT CD8⁺ T cells (p<0.001). This increase in SABG-positive cells was accompanied by elevations of CEACAM-1 (carcinoembryonic Ag-related cell adhesion molecule 1; also known as CD66a) and IL-6 (FIG. 1D) and increased expression of p53 (a cyclin-dependent kinase inhibitor) and p21 (an inducer of cellular senescence), as well as reductions of the co-stimulatory markers, CD27 (FIG. 1E and FIG. 1F) and CD28 (FIG. 1G and FIG. 1H) and (FIG. 5B), all of which are associated with activation of cellular senescence. These results indicate that premature senescence is triggered in CD8⁺ T cells deficient in eEF-2K. Furthermore, as compared with WT CD8⁺ T cells, an increased expression of PD-1 and decreased expression of CD62L were observed in eEF-2K KO CD8⁺ T cells 5 days following their activation (FIG. 5A), suggesting that ablation of eEF-2K results in an exhausted state of CD8⁺ T cells. To explore the molecular mechanisms underlying the dysfunctional status of eEF-2K KO CD8⁺ T cells, LC MS/MS analysis was performed and it was found that 1934 proteins were differentially expressed: 928 proteins in eEF-2K KO CD8⁺ T cells vs. 1006 proteins in their WT counterparts; 617 proteins remained unaffected. Moreover, comparative global proteomics analysis also revealed that the eEF-2K KO CD8⁺ T cells were resistant to apoptosis, a hallmark of senescence, and supported this hypothesis by showing higher expression of senescence markers. Together, these experiments demonstrated a critical role for eEF-2K in maintaining the robustness and function of CD8⁺ T cells, and indicate that loss of this kinase is detrimental to the functional status of CD8⁺ T cells.

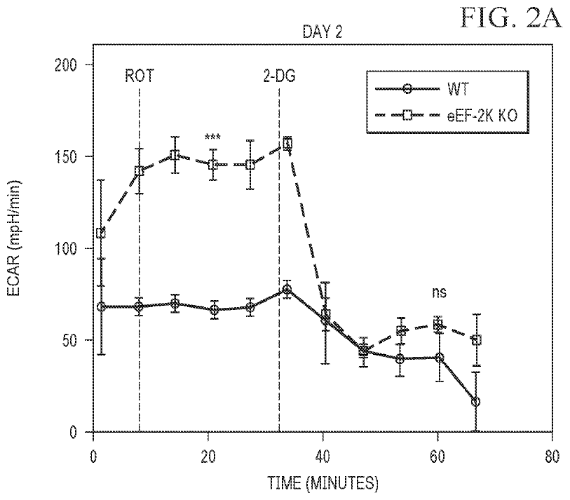

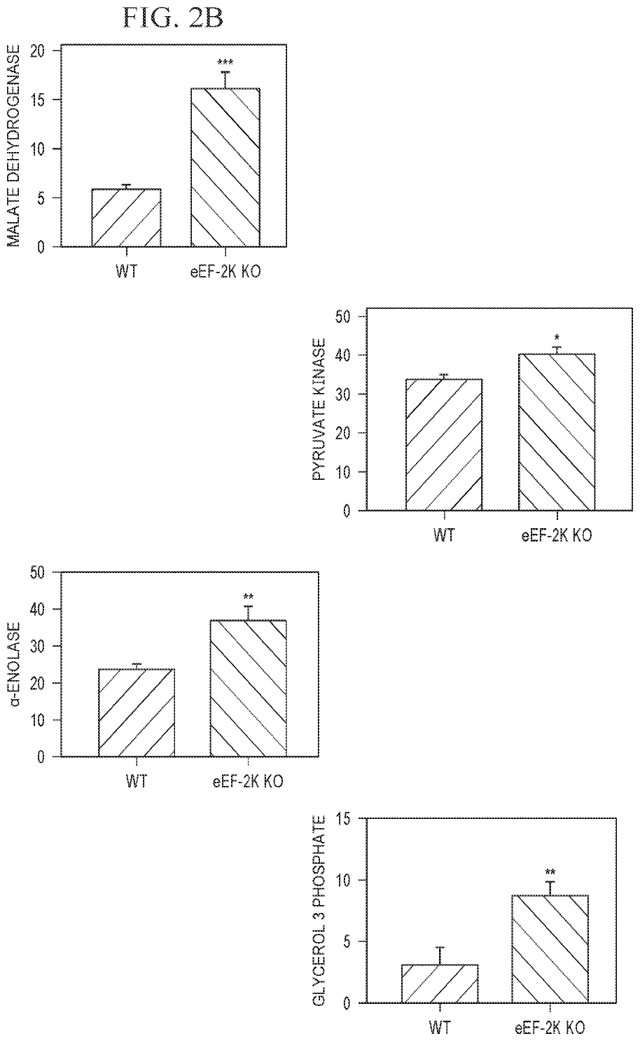

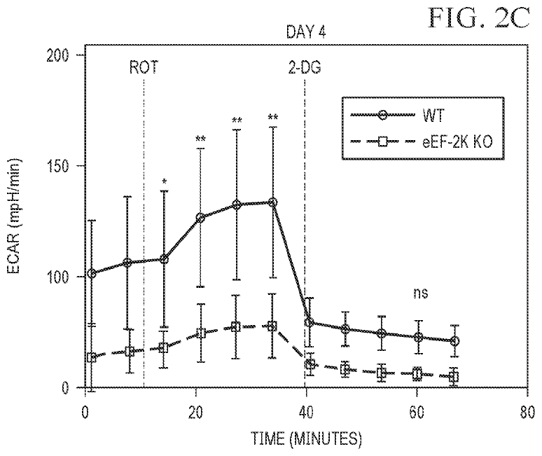

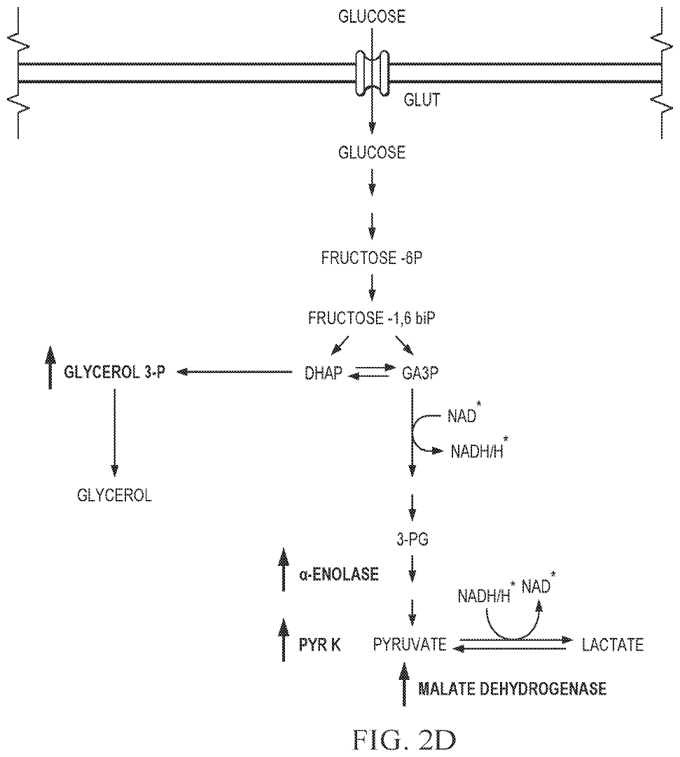

Cellular metabolism is reprogrammed in CD8⁺ T cells deficient in eEF-2K. As metabolic reprogramming is intimately associated with the differentiation, survival, and function of immune cells, and previous studies showed that expression or activity of eEF-2K has an important regulatory role in production of cellular energy, it was determined whether the impact of eEF-2K on the fate and function of CD8⁺ T cells is mediated through metabolic reprogramming. Seahorse metabolic profiling was performed to analyze cellular metabolism, and the results showed that the activated eEF-2K KO CD8⁺ T cells had a higher basal extracellular acidification rate (ECAR) than the activated WT CD8⁺ T cells (FIG. 2A), indicating that CD8⁺ T cells lacking eEF-2K are more metabolically active; nevertheless, the ECAR was decreased considerably 4 days following activation (FIG. 2C). Baseline ECAR disruptions was observed in eEF-2K KO CD8⁺ T cells metabolism 4 days post-activation, which may be attributable to alterations in metabolic homeostasis and glycolytic activity of these cells. These results suggest that the initial metabolic stress on eEF-2K KO CD8⁺ T cells may lead to their fatigue. To compare the proteome of WT CD8⁺ T cells and eEF-2K KO CD8⁺ T cells, a quantitative global proteomics analysis was performed. This analysis revealed significantly higher production of malate dehydrogenase, pyruvate kinase, alpha enolase, aldehyde dehydrogenase and glycerol 3-phosphate in eEF-2K KO CD8⁺ T cells than in control cells on day 3 post-activation (FIG. 2B), further suggesting a high glycolytic activity and metabolic exhaustion in eEF-2K KO CD8⁺ T cells (FIG. 2D). Thus, the metabolic reprograming observed in eEF-2K KO CD8⁺ T cells may account for their altered survival, proliferation, and functional status (FIG. 1A-FIG. 1H).

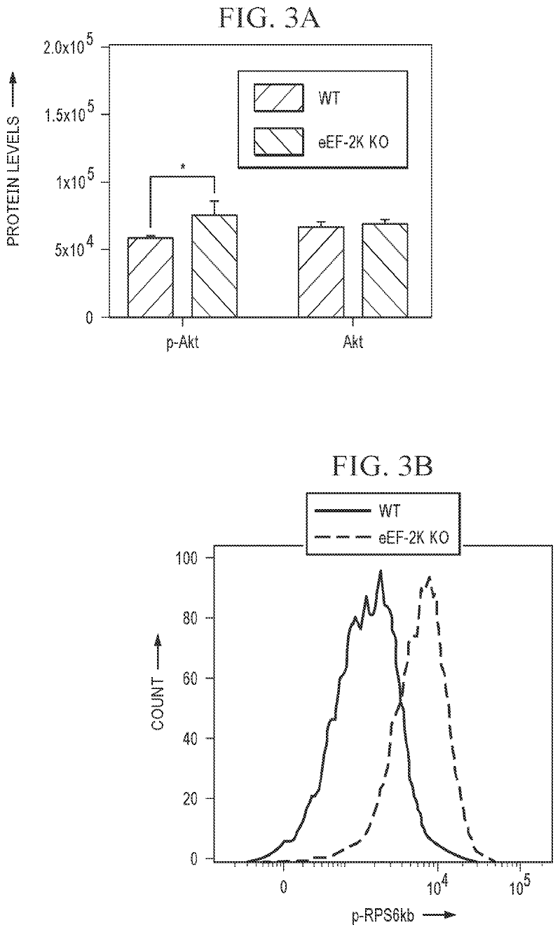

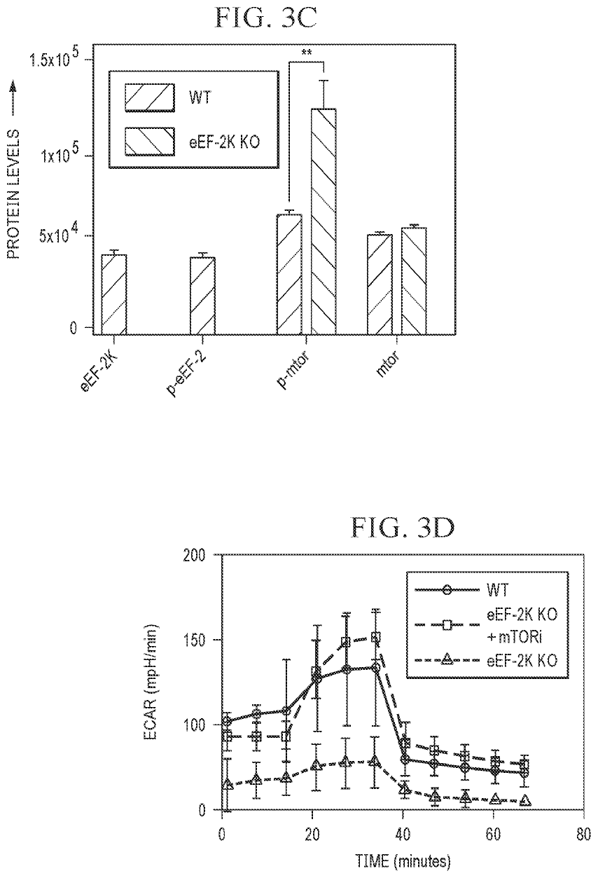

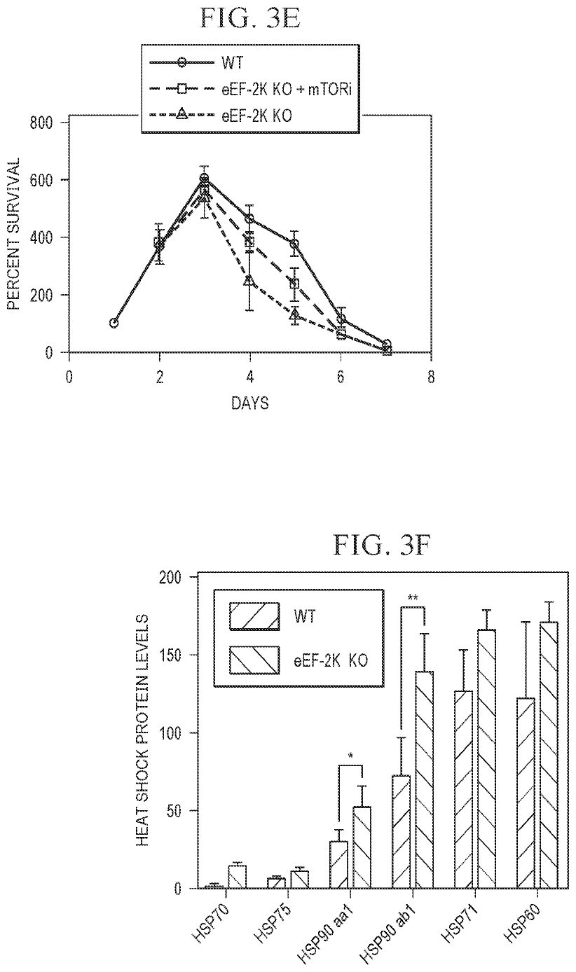

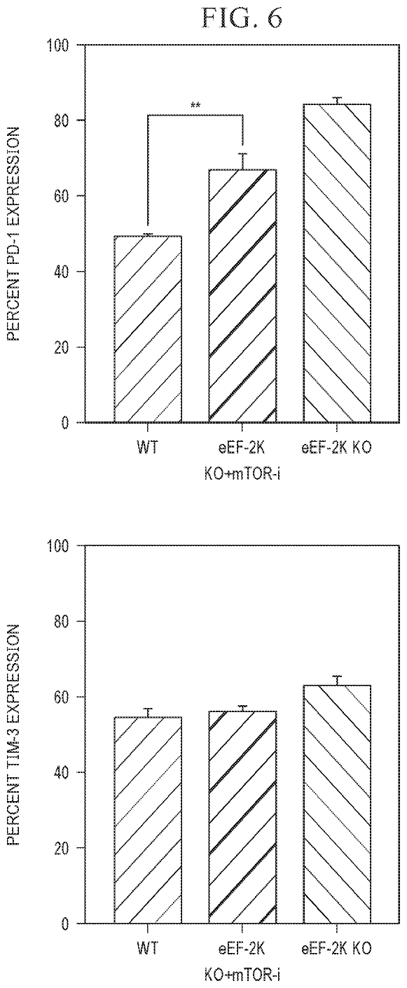

Akt-mTOR-S6K signaling is hyperregulated in CD8⁺ T cells lacking eEF-2K. It has been reported that Akt-mTORC-S6K signaling has a critical role in T cell differentiation. To determine how cellular metabolism is reprogrammed in CD8⁺ T cells lacking eEF-2K, using eEF-2K KO CD8⁺ T cells and WT CD8⁺ T cells, the activity of the Akt-mTOR-S6K signaling, which is a central pathway in cellular metabolism, proliferation, growth and survival was compared. It was found that phosphorylation of Akt, mTOR and S6K were all increased in eEF-2K KO CD8⁺ T cells compared to WT CD8⁺ T cells (FIG. 3A-FIG. 3C), suggesting that depletion of eEF-2K causes hyperactivation of the Akt-mTOR-S6K signaling, and this may be responsible for the metabolic alteration observed in CD8⁺ T cells deficient in eEF-2K (FIG. 2A-FIG. 2D). To further demonstrate the role of the higher Akt-mTOR-S6K activity in the metabolic reprogramming that occurred in eEF-2K KO CD8⁺ T cells, these cells were treated with rapamycin, a selective inhibitor of mTOR, and then analyzed ECAR. FIG. 3D shows that on day 4 post-activation, the metabolism of the rapamycin-treated eEF-2K KO CD8⁺ T cells was substantially different from that of the untreated cells but was similar to that of WT CD8⁺ T cells; this observation supported the role of the Akt-mTOR-S6K signaling in controlling CD8⁺ T cell metabolism. In addition, the survival of eEF-2K KO CD8⁺ T cells was partially improved (FIG. 3E). The expression of PD-1 and Tim-3, the proteins known to regulate immune surveillance of cancer cells, were also partially restored in the presence of rapamycin (FIG. 6) on day 6 post-activation of these cells. The differential PD-1 expression in WT CD8⁺ T cells, as shown in FIG. 5A and FIG. 6, may be attributable to the differential activation status or different time-points of data collection post-activation. These data suggest that the altered activity of the Akt-mTOR-S6K signaling in eEF-2K KO CD8⁺ T cells may play a critical role in modulating the fate and function of CD8⁺ T cells.

LC MS/MS analysis also revealed that among the differentially expressed heat shock protein 90 (HSP90) was expressed at substantially higher levels in eEF-2K KO CD8⁺ T cells than in WT CD8⁺ T cells (FIG. 3F). This finding is consistent with a previous study showing that the amounts of this chaperone protein were increased in the eEF-2K-depleted human cells and eEF-2K KO mouse embryonic fibroblasts. As the NF-κB pathway is controlled by HSP90 and can modulate the activity of Akt and mTOR, the NF-κB pathway in CD8⁺ T cells with or without ablation of eEF-2K was next analyzed. Phosphorylation of NF-κB p65 and IKKα/β subunit are critical determinants of the activity of NF-κB pathway. It was found that on day 3 post-activation, the upregulation of HSP90 in eEF-2K KO CD8⁺ T cells was accompanied by increases of phospho-NF-κB p65 and phospho-IKKα/β. Treatment of the CD8⁺ T cells with AUY-922, a small molecule inhibitor of HSP90, caused dose-dependent reductions of phospho-NF-κB p65, phospho-Akt and phospho-RPS6kb. These results suggest that the upregulation of HSP90/NF-κB induced by eEF-2K ablation may be responsible for the activation of Akt-mTOR-S6kb signaling, leading to an early enhanced metabolic state of eEF-2K KO CD8⁺ T cells.

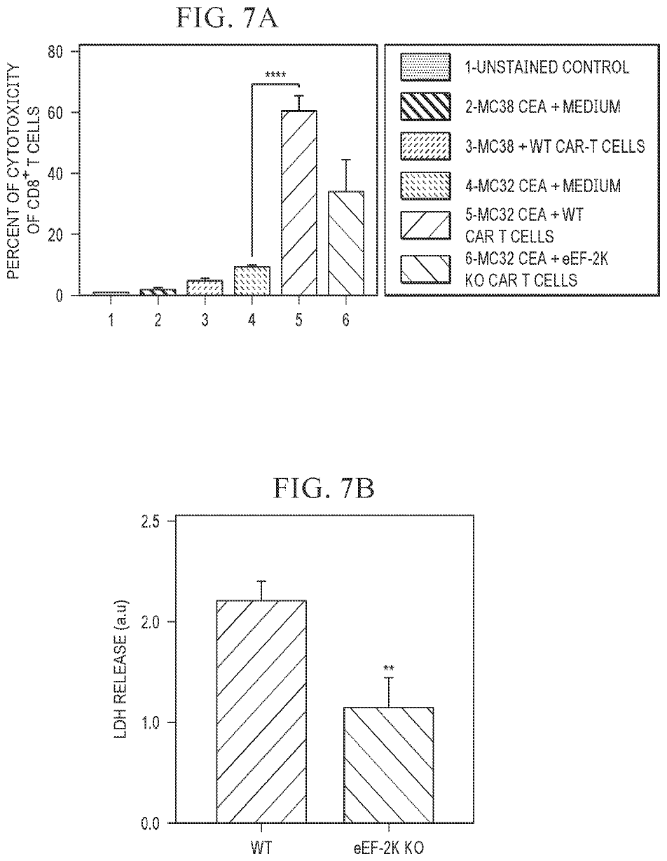

Impact of eEF-2K expression on antitumor efficacy of CAR-T therapy. To determine the importance of eEF-2K in the CD8⁺ T cell-mediated antitumor immunity, CD8⁺ T cells were isolated from the spleen and lymph nodes of WT or eEF-2K KO mice, and transduced them with a chimeric carcinoembryonic antigen (CEA) receptor construct to generate the CEA-Ag specific CD8⁺ T cells. Then, the WT CEA-specific CD8⁺ T cells or the CEA-specific eEF-2K KO CD8⁺ T cells were co-cultured with MC32 murine colon carcinoma cells expressing CEA. The cytocidal activity of CD8⁺ T cell was assessed using microscopy observation, image-cytometry analyses, and lactate dehydrogenase (LDH) release assays (FIG. 7B). The cytocidal activity of the CEA-specific eEF-2K KO CD8⁺ T cells was significantly lower than that of the WT CEA-specific CD8⁺ T cells (p=0.0009 and p=0.0002, respectively), indicating that the cytotoxicity of eEF-2K KO CEA-Ag specific CD8⁺ T cells is significantly compromised compared to WT CEA-Ag specific CD8⁺ T cells. To examine the possible off-target effects of these CAR-T cells, the CEA-specific CD8⁺ T cells were co-cultured with MC38 colon carcinoma cells not expressing CEA, and then performed identical assays as above. No off-target effects were observed in these assays (FIG. 7A).

To further demonstrate the effect of eEF-2K on cytocidal activity of CD8⁺ T cells, CD8⁺ T cells were isolated from OT-I T cell receptor (TCR) transgenic mice, transduced these cells retrovirally with an eEF-2K expression vector, and determined the effects of the overexpression of eEF-2K on the cytotoxicity and function of the CD8⁺ T cells. These OT-I CD8⁺ T cells recognize the ovalbumin (OVA) expressed on B16-OVA melanoma cells. It was shown that the cytotoxicity of the eEF-2K^{+/+} CD8⁺ T cells overexpressing eEF-2K was significantly higher than their WT counterparts (p=0.0008). Off-target effects were ruled out by co-culturing WT and eEF-2K^{+/30} CD8⁺ OT-I T cells with control B16 (OVA^{null}) melanoma cell lines. Overexpression of eEF-2K in OT-I T cells (eEF-2K^{+/+}) also improved their functional profile, as evidenced by the increased expression of CD28 and reduced expression of PD-1. In addition, some anti-inflammatory and pro-survival cytokines like TNF-α, IFN-γ, IL-2 and IL-6 were all improved in the eEF-2K^{+/+} CD8⁺ T cells. These results imply that the compromised cytotoxicity of CD8⁺ T cells can be recovered by overexpressing eEF-2K in those T cells, irrespective of the Ag-specificity of the TCR.

To recapitulate the in vitro observations in animal tumor model, mice were inoculated with MC32-CEA tumor cells (1×10⁶ cells/mouse) subcutaneously in the right lateral flank, followed by intravenous injection of WT or eEF-2K KO CEA-specific CAR-T cells (5×10⁶ cells/mouse). In this tumor model, it was observed that the tumoricidal effect of WT CAR-T cells were substantially stronger than that of eEF-2K KO CAR-T cells. All the mice receiving an i.v. infusion of WT CEA CD8⁺ CAR-T cells survived at least 28 days after tumor induction, whereas the survival of the mice treated with eEF-2K KO CAR-T cells declined rapidly from day 15 onwards. Also, the tumor-inhibitory effect of WT CEA CD8⁺ CAR-T cells was significantly weakened when eEF-2K was ablated. These results demonstrate that eEF-2K is crucial for the antitumor activity of CD8⁺ T cells.

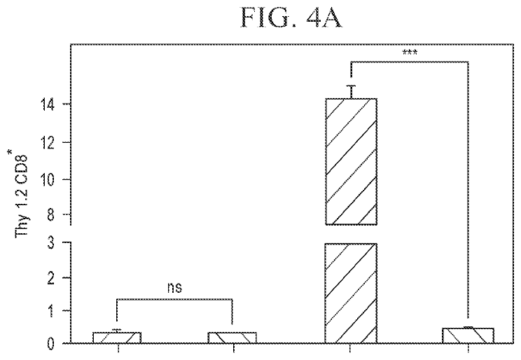

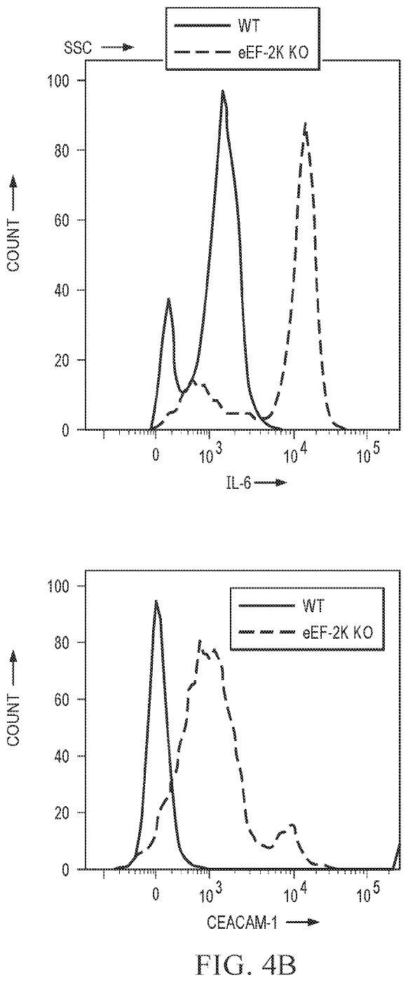

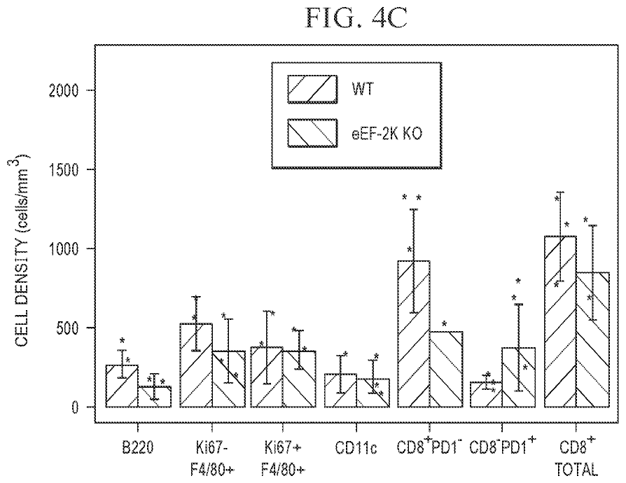

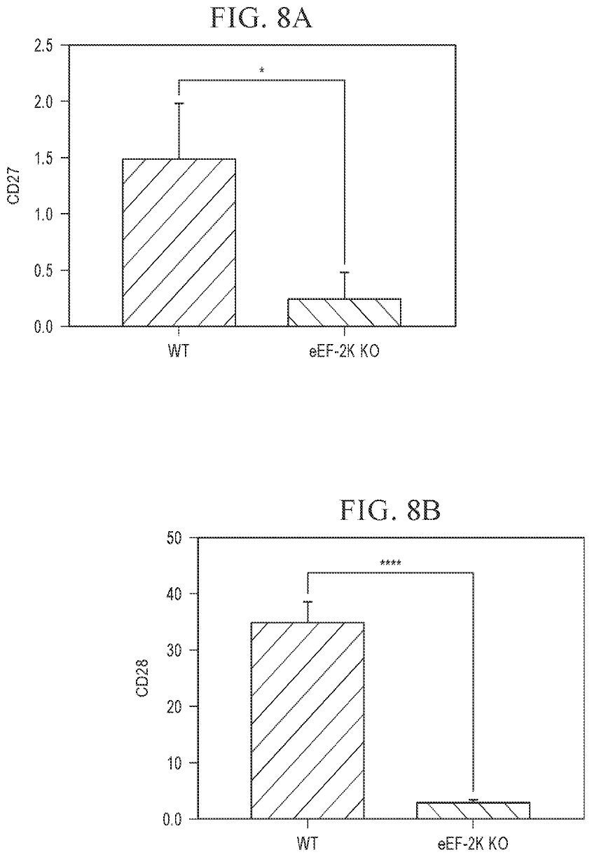

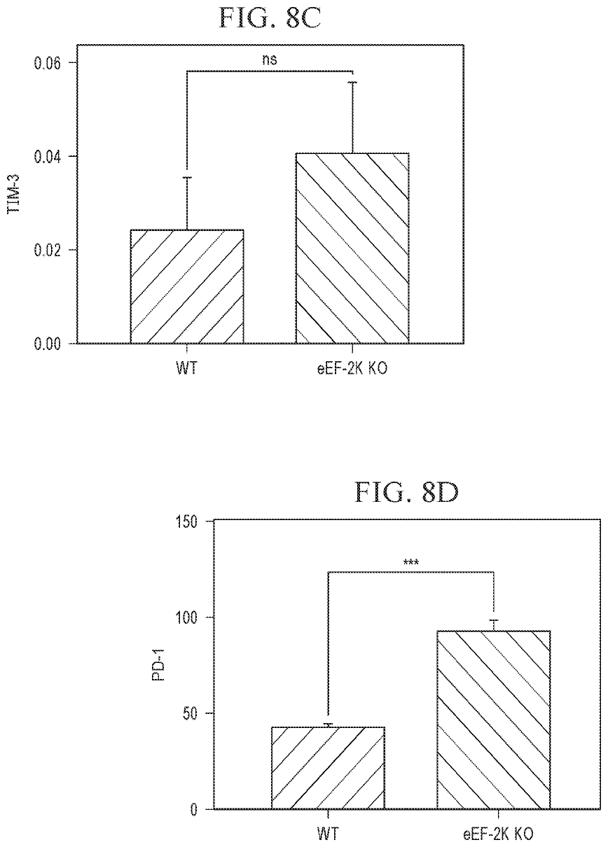

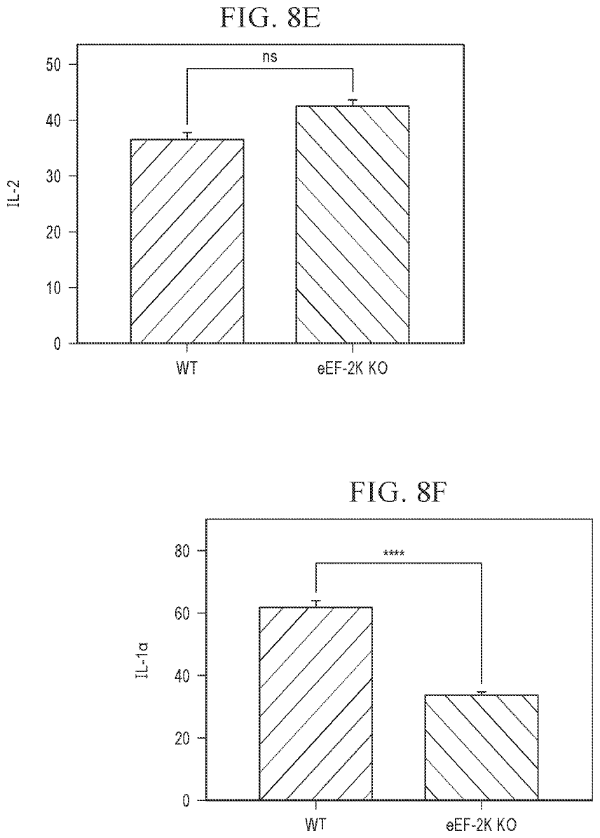

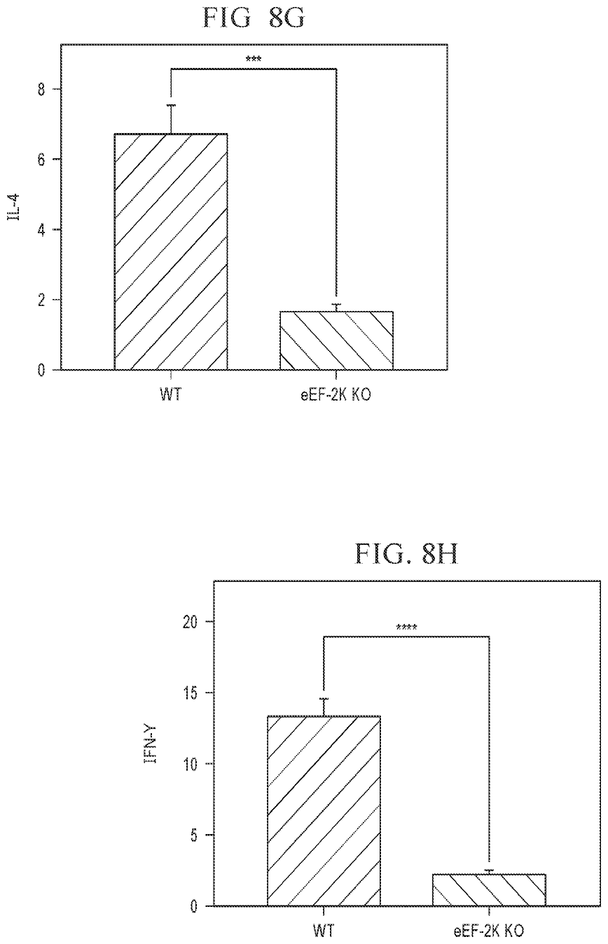

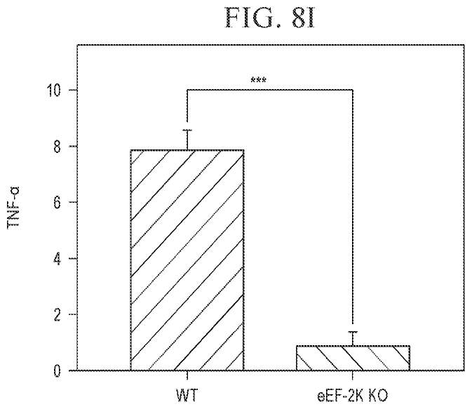

CD8⁺ CAR-T cells deficient in eEF-2K show a reduced ability to infiltrate the TME. Further, the tumor infiltration ability of the injected CAR-T cells was examined using flow cytometric analysis of the explanted tumors. Much less CEA-specific eEF-2K KO CAR-T cells were detected in the tumor tissues as compared with control cells (FIG. 4A). CEA-specific eEF-2K KO CAR-T cells had a significantly lower penetration into the TME, which was similar to the non-CEA control CD8⁺ T cells. Also, the heat map constructed from the flow cytometric dot-plots exhibited a dysfunctional state of the tumor-infiltrating eEF-2K KO CAR-T cells: compared to WT CD8⁺ CAR-T cells, the CD8⁺ CAR-T cells lacking eEF-2K produced significantly less inflammatory cytokines TNF-α, IFN-γ and IL-4, and the pro-survival cytokine IL1-α, but equivalent amount of IL-2 cytokine (FIG. 8E and FIG. 8I). In addition, the CEA-specific eEF-2K KO CAR-T cells displayed a compromised activation and high level of exhaustion, as the eEF-2K KO CAR-T cells infiltrating the TME produced more intracellular IL-6 cytokine and CEACAM-1 on their surfaces (FIG. 4B), significantly lower expression of CD27 and CD28 but significantly higher expression of PD-1 and Tim-3 and (FIG. 8A and FIG. 8D) than the WT CAR-T cells. Confocal microscopic analysis confirmed the lower penetration of eEF-2K KO CAR-T cells than WT control cells (FIG. 8J). The H&E staining of the tumor specimens showed that there were considerably greater amounts of tumor cells in the xenografts from the mice treated with the CEA-specific eEF-2K KO CAR-T cells than in the xenografts from the mice receiving the WT CEA CD8⁺ CAR-T cells. These results indicate that loss of eEF-2K not only impairs the tumoricidal activity of the CEA-specific CAR-T cells, but also weakens their ability to infiltrate tumor tissues. Furthermore, the functional profile of the tumor infiltrating CAR-T cells were analyzed and compared with or without depletion of eEF-2K. Mass cytometric analysis of the explanted tumors validated that total penetration of the WT CEA CD8⁺ T cells into TME was higher than that of CEA CAR CD8⁺ T cells with ablation of eEF-2K (FIG. 4C). In addition, the number of CD8⁺ PD-1⁺ T cells were significantly higher in the population of CEA CAR CD8⁺ T cells lacking eEF-2K, whereas the number of the CD8⁺ PD-1⁻ T cells were significantly higher in the population of the WT CEA CAR CD8⁺ T cells (p=0.1372; FIG. 4C), suggesting that exhaustion and dysfunction may account for the low penetration of CAR CD8⁺ T cells deficient in eEF-2K. Additionally, in the tumor specimens from the mice receiving the WT CEA CAR CD8⁺ T cells, an increase in Ki-67⁻ macrophages (Ki-67-F4/80⁺) was detected, whereas the tumor specimens from the mice receiving eEF-2K KO CAR-T cells had high amounts of Ki-67⁺ macrophages (Ki-67⁺ F4/80⁺) (FIG. 4C), another difference in the antitumor efficacy between the WT CEA CAR-T cells and eEF-2K KO CEA-specific CAR-T cells. The immune cells in TME were also compared using neighborhood joining heat maps and t-sne plots. The representative subtractive heatmap shows that the TME of the mice receiving eEF-2K KO CAR-T cells may be more favorable for proliferation of CD8⁺ PD-1⁻ T cells as these cells can preferably interact with Ki-67 protein. On the other hand, CD8⁺ PD-1⁻ T cells, which are functionally active and less exhausted, are less likely to interact with Ki-67 protein. The functional CD8⁺ PD-1⁻ T cells display a much lower potential of interacting with α-smooth muscle actin (α-SMA) protein, suggesting a reduced efficacy of killing tumor cells. The t-sne plot generated from the heatmap shows an overview of the TME of the mice receiving an infusion of CAR-T cells. All these results consistently demonstrate a crucial role of eEF-2K in controlling the persistence and function of cytotoxic CD8⁺ T cells.

CTLs are a key component of antitumor immunity, yet the critical determinants of their function and fate remain to be fully defined. Here, it is reported that a previously unappreciated role of eEF-2K in sustaining the survival and cytocidal activity of CTLs. The impetus for this study is the finding that eEF-2K has an important role in regulating stress responses and cellular metabolism, and the importance of metabolic reprogramming in controlling the survival, differentiation, expansion, and activation of immune cells. It is demonstrated that eEF-2K is essential for maintaining the robustness and function of CD8⁺ T cells, and that loss of this kinase is detrimental to their functional status and fate (FIG. 1A-FIG. 1H). More importantly, it was found that eEF-2K is a critical determinant of antitumor immunity of CD8⁺ T cells, as their cytocidal activity is reduced significantly both in vitro and in vivo when eEF-2K is depleted. Additionally, the ability to penetrate tumors and tumor cell-killing functions are impaired in CD8⁺ T cells deficient in eEF-2K, which could be a consequence of premature exhaustion and senescence of these cells (FIG. 1A-FIG. 1H). It is further shown that cellular metabolism is altered in the eEF-2K-deficient CD8⁺ T cells (FIG. 2A-FIG. 2D), which is associated with hyper-activation of the Akt-mTOR-S6K pathway (FIG. 3A-FIG. 3F). Thus, the metabolic reprogramming mediated by eEF-2K may account for the effects of eEF-2K on CD8⁺ T cells.

As protein synthesis is one of the most notable consumers of cellular energy and eEF-2K is a key regulator of protein synthesis and a critical checkpoint in energy consumption, deficiency of this kinase may lead to metabolic catastrophe. Therefore, the alterations of cell proliferation, survival, senescence and function (FIG. 1A-FIG. 1H) as well as metabolic activity (FIG. 2A-FIG. 2D) observed in eEF-2K KO CD8⁺ T cells could be a cellular response to metabolic stress, which is critical for the sustained activity and survival of CD8⁺ T cells. Nevertheless, excessive stress responses can negatively affect cell fate, as evidenced by premature senescence, shorter lifespan and weakened function of CD8⁺ T cells deficient in eEF-2K (FIG. 1A-FIG. 1H). Indeed, the premature and dysfunctional state of eEF-2K KO CD8⁺ T cells (FIG. 1A-FIG. 1H), which likely results from the metabolic reprogramming following activation, significantly weakens their cytocidal activity, tumoricidal action and tumor infiltration ability. These experiments could differentiate the tumor infiltrating lymphocytes from the tissue resident CD8⁺ T cells, but were barely able to distinguish the infiltration and expansion of these cells within the TME. The impact of eEF-2K expression on the cytocidal activity of CD8⁺ T cells was further validated by the experiments demonstrating that augmenting eEF-2K in tumor Ag-specific CD8⁺ T cells enhanced the antitumor efficacy of CTLs and improved their functionality.

The altered metabolic status of eEF-2K KO CD8⁺ T cells was manifested by higher glycolytic activity (FIG. 2A-FIG. 2D) and upregulated Akt-mTOR signaling following activation (FIG. 3A-FIG. 3C). The enhanced Akt-mTOR activity in eEF-2K KO CD8⁺ T cells is likely caused by HSP90 upregulation (FIG. 3F) and NF-κB activation, as inhibition of HSP90 by AUY-922, a small molecule inhibitor of HSP90, is accompanied by downregulation of the Akt-mTOR signaling. These results are consistent with a recent report showing that the expression of the metabolic signaling proteins, including NF-κB, mTOR and pRPS6kb, were upregulated in the early-activated CD8⁺ T cells as a way of metabolic adaptation. Recently, it has also reported that maintaining the equilibrium of the Akt-mTOR signaling pathway is critical for promoting T cell quiescence, longevity, and homeostasis. In addition, elevations of HSP90 and the phospho-Akt proteins have been reported in other types of cells deficient in eEF-2K, including the intestinal stem cells, epithelial cells, human tumor cells, and mouse embryonic fibroblasts. Similarly, it was found that the upregulations of these signaling also occur in CD8⁺ T cells subjected to eEF-2K ablation (FIG. 3A and FIG. 3F). It would be interesting to determine whether the activities of those signaling in other subsets of T cells or immune cells are also affected by eEF-2K expression. In addition, previous studies have shown that Akt is involved in the control of the metabolic fate of CD8⁺ T cells via T cell receptor (TCR) signaling and that mTOR activity is upregulated in the highly proliferative effector CD8⁺ T cells. However, whether eEF-2K expression or activity is lost or reduced in those CD8⁺ T cells remain to be investigated.

In summary, this study identifies eEF-2K as a crucial regulator of the antitumor immunity of CTLs. eEF-2K is essential for the viability and function of those CD8⁺ T cells, and the effects of this kinase on these T cells are mediated through the Akt-mTOR-S6K pathway. Furthermore, it is demonstrated that the regulation of CD8⁺ T cells by eEF-2K significantly impacts their antitumor function and ability to penetrate the TME. Thus, the critical role of eEF-2K in upholding the activity and function of CTLs warrants further investigation to assess whether therapeutic augmentation of this kinase can be exploited as a novel approach to reinforcing CAR-T therapy against cancer.

Reference will now be made to particular materials and methods utilized by various embodiments of the present disclosure. However, it should be noted that the materials and methods presented below is for illustrative purposes only and is not intended to limit the scope of the claimed subject matter in any way.

Cell lines and culture. Murine colon adenocarcinoma cells (MC38, CEA⁻) or MC32 (MC32, CEA⁺) were grown in DMEM medium with 10% FBS, 1% L-Glutamine and 1% Penicillin-streptomycin. The cells were grown to confluence in 5% CO₂ incubators and used for in vitro murine CD8⁺ T cell co-culture and in vivo solid tumor induction experiments. B16 or B16-OVA melanoma cells were also grown in DMEM medium with 10% FBS, 1% L-Glutamine and 1% Penicillin-streptomycin and maintained in 5% CO₂ incubators.

Global proteomics analysis. The sample preparation for liquid chromatography-mass spectrometry/mass spectrometry (LC-MS/MS) analysis was performed by using a previously described in-gel digestion methodology for sample preparation from WT and eEF-2K KO CD8⁺ T cells cultured for 3 days post-activation with anti-CD3/CD28 antibodies, with minor modifications. The spectral analysis was performed by extracting the tandem mass spectra and all MS/MS samples were analyzed using Mascot (Matrix Science, London, UK; version 2.7.0) and X Tandem (2010.12.01.1). Tandem was set up to search a reverse concatenated subset of the contaminants_20120713_UniProt_Mouse_20161004 database with 21478 entries. Tandem were searched with a fragment ion mass tolerance of 0.80 Da and a parent ion tolerance of 20 PPM. Carbamidomethyl of cysteine was specified in Mascot and Tandem as a fixed modification. Deamidation of asparagine and glutamine, oxidation of methionine, acetyl of the n-terminus and phosphorylation of serine, threonine and tyrosine were specified in Mascot as variable modifications. The total differentially expressed proteins in WT vs eEF-2K KO CD8⁺ T cells were enumerated from mass spectrometric analysis results and plotted in a Venn-diagram. Comparative protein amounts of Ki-67, Malate dehydrogenase, Pyruvate kinase, Glycerol-3-phosphate, Alpha Enolase, apoptotic and senescent marker proteins as well as heat shock proteins were enumerated from the data and plotted graphically.

Animal experiments. eEF-2K KO mice (C57BL/6 background; Thy1.2⁺) were generated previously. C57BL/6 congenic mice (B6 Thy1.2; Thy1.1⁺) were obtained from The Jackson laboratory (Bar Harbor, ME) and maintained in-house in specific pathogen free (SPF) BSL2 facility with 12:12h light-dark cycle. Naive CD8⁺ T cells were isolated from the pooled splenocytes and lymph nodes of the WT and eEF-2K KO mice using the negative CD8⁺ T cell selection with Mojosort Mouse T cell isolation kit (BioLegend, San Diego, CA). The CD8⁺ T cells harvested were used for both in vitro and in vivo adoptive immunotherapy experiments.

C57BL/6 congenic mice (6-8 weeks old, male/female) were used for adoptive tumor immunotherapy experiments. The mice were divided into 4 different groups (n=5) and maintained in SPF-BSL2 facility at 12:12h light-dark cycle, 68° F. to 72° F. ambient temperature and 30% to 70% humidity for tumor immunotherapy experiments.

OT-I TCR transgenic mice (6-8 weeks old) were used to isolate CD8⁺ T cells for overexpression of eEF-2K. These mice contain transgenic inserts for mouse Tcra-V2 and Tcrb-V5 genes in CD8⁺ T cells. The OVA-specific TCRs on CD8⁺ T cells recognize MHC class I-restricted OVA epitope on B16-OVA melanoma cells. The WT or eEF-2K overexpressing OT-I CD8⁺ T cells were used in in-vitro co-culture assay with B16-OVA melanoma cells. All animal studies were conducted in accordance with the guidelines of Institutional Animal Care and Use Committee (IACUC #2018-0065), Texas A&M University.

Overexpression of eEF-2K in OT-I CD8⁺ T cells. eEF-2K gene was cloned from pCDNA3-HA-eEF2K (Addgene #110160) vector and inserted into the gamma-retroviral vector pMIG modified from the backbone pMSCV 2.2. The pMIG vector was first transduced into platinum-E (Plat-E) retroviral packaging cell line allowing retroviral packaging with a single plasmid transfection. The viral supernatants were then used to transduce the OT-I CD8⁺ T cells to generate eEF-2K-overexpressing T cells (eEF-2K^{+/+}). A mock transduction with empty vector was also performed as a control.

CAR-T cell preparation. The murine stem-cell-based gamma-retroviral vector MSGV1, which was used as a control vector in this study, is composed of CAR elements of CD28 and CD3z moieties but lacks CEA-Ag expression. MSGV4 retroviral vector was modified from the MSGV1 background to express CEA-Ag specific scFv with other CAR elements derived from MSGV1 backbone. WT and eEF-2K KO CD8⁺ T cells were transduced with the viral supernatants containing MSGV1 (control; no CEA) or MSGV4 (CEA). Briefly, naive CD8⁺ T cells isolated from WT and eEF-2K KO B6 Thy1.2 mice were stimulated and maintained in RPMI medium (10% FBS, 50 μM 2-Mercaptoethanol, 1% Penicillin-Streptomycin, 1% NEAA, 1% Sodium Pyruvate and 50 U/ml IL-2).

Retroviral supernatants produced from MSGV1-null or MSGV4-CEA transduced Plat-E packaging cell line was added to the isolated WT and eEF-2K KO CD8⁺ T cells in RPMI medium supplemented with 5 μg/ml Polybrene (Sigma Aldrich, San Louis, MO). The cells were then centrifuged at 32° C. for 1 hour and further incubated at the same temperature in 5% CO₂incubators for 6 hours. The transduced CD8⁺ T cells were identified by analyzing c-myc expression in BD Fortessa X-20 flow cytometer (BD Bioscience, San Jose, CA).

Assays for survival, proliferation, and IL-2 production of CD8⁺ T cells. Naive WT and eEF-2K KO CD8⁺ T cells isolated from B6 Thy1.2 mice were activated by anti-mouse CD3 antibody (Ab, clone 2C11; BioLegend, San Diego, CA)/anti-mouse CD28 Ab (clone 37.51; BioLegend, San Diego, CA) and monitored for their survival by trypan-blue cell exclusion method using TC20 automated cell counter (Bio-Rad, USA). The live CD8⁺ T cells were counted and plotted graphically with GraphPad Prism 9. CD8⁺ T cell proliferation was measured by carboxy-fluorescein succinimidyl ester (CFSE; Invitrogen, Carlsbad, CA) assay. WT and eEF-2K KO CD8⁺ T cell IL-2 secretion was assessed from Day 1 and Day 3 cell culture supernatants using enzyme-linked immunosorbent assay (ELISA) kits (BioLegend, San Diego, CA) as per manufacturer's instructions.

Western blots. WT and eEF-2K KO CD8⁺ T cells were lysed with RIPA lysis buffer, 30 μg of protein lysate was tested for SDS-PAGE. Briefly, proteins resolved by 10% SDS-PAGE gel were transferred onto PVDF membranes with a semi-dry electroblotting system. The PVDF membranes were then blocked for 1h at room temperature in 2% BSA and subsequently probed with primary antibodies for eEF-2K (cat. no. 3692; Cell signaling, Danvers, MA), p-Akt (S472; clone no. 104A282; BD Bioscience, San Jose, CA), Akt (clone no. 094E10 BioLegend, San Diego, CA), p-mTOR (S2448; cat. no. 2971 Cell signaling, Danvers, MA), mTOR (cat. no. A301-143A; Bethyl Lab, Montgomery, TX) and phospho-RPS6kb (clone no. A17020B; S235/S236; BioLegend, San Diego, CA). The membranes were then washed and probed with appropriate HRP-conjugated secondary antibodies (Cell signaling, Danvers, MA) as required. The blots were stripped and re-probed with R-actin Ab which served as the loading control.

Assay for senescence-associated β-galactosidase (SA-βgal) activity. WT and eEF-2K KO CD8⁺ murine T cells were isolated from WT and eEF-2K KO B6 Thy1.2 mice and cultured in RPMI medium, until 8 days post-activation with anti-CD3/CD28 antibodies. The senescence of WT CD8⁺ T cells or eEF-2K KO CD8⁺ T cells was then compared by performing SA-β gal staining (#CBA-230, Cell biolabs, San Diego, CA) as per the manufacturer's protocols. SA-βgal positive senescent cells stain blue green in the assay. The senescent CD8⁺ T cells were imaged and quantified with Leica Slide Scanner microscope.

Adoptive cell transfer. C57BL/6 congenic mice were subcutaneously injected with 1×10⁶ MC32 CEA tumor cells in the right lateral flank on Day 0. Following tumor injection, the mice were divided into 5-different groups. CD8⁺ T cells were isolated from WT and eEF-2K KO B6 Thy1.2 mice on Day 5 and retrovirally transduced with either MSGV4 (CEA) or MSGV1 CAR constructs. T cells were cultured for 2 more days post transduction and subsequently i.v. infused into different tumor bearing mice. Untreated tumor bearing mice did not receive any infusion of CD8⁺ T cells and served as the control group. The mice were monitored for survival and tumor size up to Day 28 post tumor induction. The experiment was terminated on Day 28 and the explanted tumor was analyzed by flowcytometry, image mass cytometry, and confocal microscopy as described in methodology sections.

Comparative metabolic profiling. The glycolytic states of WT and eEF-2K KO CD8⁺ T cells were analyzed by using extracellular flux (XF) analyzer (Agilient) using manufacturer's protocol with modifications. Briefly, T cells were activated with anti-CD3/CD28 antibodies and cultured for 2 to 4 days in RPMI medium before assay. Subsequently, 1×10⁵ WT or eEF-2K KO T cells were removed from suspension from 48-well plates and transferred to 96-well poly-lysine coated Seahorse XF96 Cell Culture Microplate in Phenol Red-free RPMI-based assay media. The plate was centrifuged to facilitate the attachment of T cells and then the ECAR was measured following the manufacturer's protocol.

Flow cytometric analysis. In vitro cultured CD8⁺ T cells or explanted tumor sections were analyzed by flow cytometry. For intracellular T cell cytokine staining analysis, CD8⁺ T cells were isolated from the explanted tumor and cultured in the RPMI medium for 4 days, prior to flowcytometric analysis. Mouse CD8⁺ T cells in in vitro culture were stained with fluorochrome conjugated anti-PD-1, anti-CD27, anti-CD28, Tim-3 (clone nos. 29F.1A12; RMT3-23; LG 3A10; 37.51 respectively; BioLegend, San Diego, CA). NF-κB signaling was assessed using antibodies (pIkκ-α/β, p-NF-kb p65, NF-κB p65, Ikκ-α/β) from NF-κB pathway sampler kit (cat. no. #9936; Cell Signaling, Danvers, MA). The dead cells were excluded from analysis by using Aqua Zombie NIR staining dye (BioLegend, San Diego, CA) and gated. For in vivo CAR-T cell-based tumor inhibition studies, the explanted tumor was homogenized into single cell suspension using GentleMACS mouse tumor dissociation kit (Miltenyi Biotech, Auburn, CA). The tumor infiltrating lymphocytes were then analyzed for their functional profile and infiltration using fluorochrome conjugated anti-mouse Thy1.2 (CD90.2; BD Biosciences, San Jose, CA), anti-PD-1, anti-CD27, anti-CD28, anti-Tim 3 antibodies (BioLegend, San Diego, CA). The tumor-infiltrating lymphocytes were also sorted using the BD FACS Aria and the intracellular cytokine staining of the ex vivo activated CD8⁺ T cells was performed. Fluorochrome conjugated anti-TNF-α (clone no. MP6-XT22), anti-IFN-γ (clone no. XMG1.2), anti-IL-4 (clone no. 11B-11), anti-IL-1α, and anti-IL-2 antibodies (clone no. JES6-5H4) were used for intracellular cytokine staining analysis. All data were acquired using BD Fortessa X-20 flow cytometer (BD Bioscience, San Jose, CA) with FACSdiva v8 interface.

The data was interpreted and analyzed using FlowJo v10.7. The imaging flow cytometer analysis for in-vitro dead-live assay was acquired using Amnis Imagestream imaging flow cytometer (Luminex Corp, USA). The WT and eEF-2K KO CAR-T cells were stained with CFSE dye and co-cultured with MC32 CEA cells. The cells were stained with aqua zombie dye post-assay for determining the live and dead cells which were represented by pseudo-colored image representation. Graphs were constructed and statistically analyzed in GraphPad Prism 9.

Tumor imaging and immunohistochemistry. Fresh solid tumor samples were paraffin embedded and sliced into 4 μm sections with microtome. The prepared slides were processed for hematoxylin and eosin (H&E) staining, fluorescence microscopy and mass-cytometry analysis. The H&E section scoring was done on a scale of 1 to 5, 1 being the least tumor inhibition and 5 being the highest tumor inhibition. The tumor inhibition was calculated by considering the parameters for the number of infiltrating cells into the tumor and total of tumor cells in the xenografts for WT and eEF-2K KO CD8⁺ T cell treated mice.

Imaging mass cytometry (IMC) analysis. Mass cytometry utilizes heavy metal label conjugated antibodies, greatly enhancing the deep immunophenotyping analysis of tumor samples. A dimensionality reduction technique t-Distributed Stochastic Neighbor Embedding (t-SNE) was used to analyze several different tumor-associated immune cell markers among the WT CEA-CAR CD8 and eEF2K KO CEA-CAR CD8 infused groups of mice. Heatmap plots of the number of cells per neighborhood across the imaged tumor samples were constructed to analyze the local cell densities within individual neighborhoods as described previously. A subtractive heatmap was constructed from the data to show the differential immune cell relationships among the cell types. Ir191, Er167, Dy162, Er170Sm149 and Yb176 were used for staining DNA, Ki-67 antigen, CD8⁺ T cells, B220 (B cells), CD11b (dendritic cells) and F4/80 (macrophages) respectively.

Statistical Analysis. Multiple student's unpaired t-test or 1-way/2-way ANOVA was performed to analyze the differences between the groups. For mice survival curve analysis Kaplan-Meier method was adopted and compared statistically using log rank test in GraphPad Prism. A P-value of less than 0.05 was considered significant. Illustrations and schematic representations in figures are created by using the BioRender software.

As outlined above, eEF2K, a key regulator of protein synthesis, has important roles in modulating stress responses and cellular metabolism. As shown herein eEF2K plays an important role in regulating the fate and cytocidal activity of CD8+ T cells. It was demonstrated that CD8⁺ T cells from eEF-2K KO mice were more proliferative, but had lower survival than their wild-type counterparts after their activation, followed by the occurrence of premature senescence and exhaustion. eEF2K KO CD8⁺ T cells were identified to be more metabolically active and showed hyperactivation of the Akt-mTOR-S6K pathway. Loss of eEF2K significantly impaired the cytocidal activity of CD8⁺ T cells against tumor cells. Further, in a murine colon adenocarcinoma model, the antitumor efficacy and tumor infiltration of the CAR-CD8⁺ T cells lacking eEF2K were remarkably reduced as compared to the control CAR-CD8⁺ T cells. These results indicate that eEF2K is required for sustaining the viability and function of cytotoxic CD8⁺ T cells, and suggest that therapeutic augmentation of this kinase may be exploited as a novel approach to reinforcing CAR-T therapy against cancer and infectious diseases.

TABLE 1
Antibodies used in western blot and flow cytometric analysis.

| Antibodies | | | |
|---|---|---|---|
| (Western Blot) | Cat. No. | Secondary | Company |
| Akt | 680302 | Anti-mouse HRP | BioLegend |
| p-Akt | 550747 | Anti-mouse HRP | BD Bioscience |
| mTOR | A301-143 A | Anti-rabbit HRP | Bethyl lab |
| p-mTOR | 2971 | Anti-rabbit HRP | Cell Signaling |
| p-RPS6Kb | 608602 | Anti-mouse HRP | BioLegend |
| eEF-2K | #3692 | Anti-mouse HRP | Cell signaling |
| Antibodies | | | |
| (Flowcytometry) | Cat no. | Fluorophore | Company |
| PD-1 | 135210 | APC | BioLegend |
| Tim-3 | 119727 | BV-711 | BioLegend |
| CD27 | 124210 | PE | BioLegend |
| CD28 | 102127 | BV 421 | BioLegend |
| IL-2 | 503810 | APC | BioLegend |
| IL-1-α | 14-7011-81 | FITC | e-Bioscience |
| IFN-γ | 505805 | FITC | BioLegend |
| TNF-α | 506327 | BV-421 | BioLegend |
| IL-4 | 504103 | PE | Biolegend |
| CD8 | 300906 | FITC | BioLegend |
| CD90.2 (Thy 1.2) | 561974 | APC | BD Biosciences |
| NF-KB sampler kit | 9936 | Various | Cell signaling |

TABLE 2
Summary of abbreviations.
Abbreviations

eEF-2K: Eukaryotic elongation factor-2 kinase
KO: knockout
CAR: chimeric antigen receptor
TME: the tumor microenvironment
WT: wild type
ECAR: extracellular acidification rate
HSP90: heat shock protein 90
CEA: carcinoembryonic antigen
TCR: T cell receptor
CFSE: carboxy-fluorescein succinimidyl ester
Ab: antibody
Ag: antigen
SA-β gal: senescence-associated β-galactosidase
H&E: hematoxylin and eosin
t-SNE: t-Distributed Stochastic Neighbor Embedding

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

What is claimed is:

1. A method of reducing cytocidal activity of cells, the method comprising:
altering eukaryotic elongation factor-2 kinase (eEF-2K) in a subject; and
inducing a reduction of cytocidal activity of cytotoxic T cells (CTLs) in the subject.

2. The method of claim 1, further comprising regulating, by the altering of eEF-2K in the subject, stress responses or cellular metabolism in the subject.

3. The method of claim 1, wherein the CTLs are CD8⁺ or CD4⁺ cells.

4. The method of claim 1, further comprising inducing metabolic reprograming in the subject.

5. The method of claim 4, further comprising controlling a function of immune cells in the subject selected from the group consisting of survival, differentiation, expansion, activation, and combinations thereof.

6. The method of claim 1, wherein the altering of eEF-2K in the subject comprises ablation of eEF-2K and thereby results in an exhausted state of cytotoxic T cells in the subject.

7. The method of claim 1, wherein the altering of eEF-2K in the subject comprises an increase in phosphorylation of at least one of Akt, mTOR, or S6K to thereby deplete eEF-2K.

8. The method of claim 7, wherein the depleted eEF-2k induces hyperactivation of Akt-mTOR-S6K signaling.

9. The method of claim 1, wherein the altering of eEF-2K in the subject comprises a decrease of eEF-2K in the subject.

10. The method of claim 9, wherein the decrease reduces survival and function of cytotoxic T cells.

11. The method of claim 9, wherein the decrease alters at least one of cell proliferation, premature cellular senescence, or exhaustion thereby reducing survival and function of cytotoxic T cells.

12. The method of claim 9, wherein the decrease activates Akt-mTOR-S6K signaling and reprogrammed metabolism thereby reducing survival and function of cytotoxic T cells.

13. The method of claim 12, wherein Akt-mTOR-S6K signaling is caused by an uptake of heat shock protein 90.

14. The method of claim 12, wherein Akt-mTOR-S6K signaling is caused by NF-κB activation.

15. The method of claim 1, wherein the altering of eEF-2K in the subject comprises therapeutic augmentation of this kinase to reinforce chimeric antigen (Ag) receptor (CAR) T-cell therapy.

16. The method of claim 1, wherein the subject is being treated for cancer.

17. The method of claim 1, wherein the subject is being treated for an infectious disease.

18. The method of claim 1, wherein the altering of eEF-2K in the subject impairs activity of cytotoxic T cells in the subject.

19. The method of claim 1, wherein the altering of eEF-2K in the subject impairs function of cytotoxic T cells in the subject.

20. The method of claim 1, wherein the altering of eEF-2K in the subject reduces survival of cytotoxic T cells in the subject.
